(19) United States
(12) Patent Application Publication
(10) Pub. No.: US 2011/0014174 A1
(43) Pub. Date: Jan. 20, 2011

(54) Lysozyme, A Novel Vasodilator of Sepsis that can be inhibited by a Peroxidase and Hydroxyl Radical Scavengers

(75) Inventors: Steven Mink, Winnipeg (CA); Krika Kasian, Winnipeg (CA); Hans Jacob, Winnipeg (CA); Robert Bruce Light, Winnipeg (CA)

Correspondence Address: ADE & COMPANY INC., 2157 Henderson Highway, WINNIPEG, MB, R2G1P9, CA

(73) Assignee: UNIVERSITY OF MANITOBA, Winnipeg, MB (CA)

(21) Appl. No.: 12/675,672

(22) PCT Filed: Aug. 28, 2008
(86) PCT No.: PCT/CA2008/001513

§ 371 Date: Oct. 1, 2010

Related U.S. Application Data
(60) Provisional application No. 60/968,391, filed on Aug. 28, 2007.

(51) Int. Cl. A61K 38/44; A61K 31/5415; A61K 31/05; A61K 31/047; A61K 31/4985; A61K 31/47; A61P 9/08

(52) U.S. Cl. 424/94.4; 514/226.2; 514/731; 514/738; 514/250; 514/311

(57) ABSTRACT

A method of reducing a vasodilatory response comprising administering to an individual in need of such treatment an effective amount of a guanylate cyclase pathway inhibitor selected from the group consisting of methylene blue, ODQ, LY83583 and a protein kinase G inhibitor or an H₂O₂ reducing agent selected from the group consisting of Aspergillus niger catalase and hydroxyl scavengers, hydroquinone and mannitol.

### PRIOR APPLICATION INFORMATION

The instant application claims the benefit of U.S. Provisional Patent Application 60/968,391, filed Aug. 28, 2007.

### BACKGROUND OF THE INVENTION

Septic shock is a clinical syndrome that results from an activated systemic host inflammatory response to infection leading to cardiovascular collapse. (1) Widespread systemic vasodilation and a decrease in myocardial contractility contribute to the hypotension observed in this condition (1,25,26,28). Both components reflect the effects of mediators that are released as part of the inflammatory response. The recruitment of leukocytes into sites of inflammation leads to interaction with the vascular endothelium and promotes the release of a wide range of proinflammatory cytokines, such as interleukin-6 and tumor necrosis factor-α among other classes of mediators (12,28). Many of these mediators are associated with the release of nitric oxide (NO) by activation of endothelial and inducible nitric oxide synthases. NO is thought to play a prominent role in the vasodilation that accompanies septic shock (10).

We previously found that lysozyme (Lzm-S), released from leukocytes contributed to the myocardial depression that develops in septic shock (13,19-21). We showed that the mechanism by which Lzm-S caused myocardial depression was related to the binding of its catalytic site to the endocardial endothelium (EE) (19). This interaction resulted in the release of NO, the effect of which could be inhibited by the non-specific nitric oxide synthase (NOS) inhibitor N^{G}-monomethyl-L-arginine (L-NMMA). After release of NO from the EE, NO diffuses to adjacent myocytes to activate the guanosine 3′,5′ monophosphate (cGMP) pathway. cGMP may then depress contraction by multiple mechanisms that include among others a reduction in myofilament Ca²⁺ responsiveness via activation of cGMP-dependent protein kinase (PKG), and an inhibition of myocardial calcium channel activity (17).

Since Lzm-S causes NO release by interaction with the EE, we hypothesized that Lzm-S may also contribute to the systemic vasodilation observed in septic shock by binding to the endothelium of the systemic vasculature. In the present study, we used a phenylephrine contracted carotid artery ring preparation to examine the extent to which Lzm-S may contribute to a reduction in vasculature tone in septic shock. We examined whether release of NO by the endothelium or whether other mechanisms contributed to Lzm-S's vasodilatory response.

### SUMMARY OF THE INVENTION

According to a first aspect of the invention, there is provided a method of reducing a vasodilatory response comprising administering to an individual in need of such treatment an effective amount of a guanylate cyclase pathway inhibitor or an H₂O₂ reducing agent.

### BRIEF DESCRIPTION OF THE DRAWINGS

FIG. 1. In the examples shown, canine lysozyme was placed into the phenylephrine constricted carotid ring preparation in Panel A, while human lysozyme was placed into the preparation in Panel B. There was a decrease in constriction to about 40% of the phenylephrine value in both conditions.

FIG. 2. In the graphs shown, the mean values are shown for the canine lysozyme experiment in Panel A and for the human lysozyme experiment in Panel B. Both lysozyme preparations caused significant vasorelaxation in the carotid artery preparation.

FIG. 3. Examples in which treatment with the non-specific nitric oxide inhibitor L-NMMA (N^{G}-monomethyl-L-arginine) (Panel A) and removal of the endothelium by mechanical denudation (Panel B), respectively, did not inhibit lysozyme induced vasodilation.

FIG. 4. In the example shown, incubation with methylene blue inhibited the effect of lysozyme.

FIG. 5. Inhibitors of the guanosine 3′,5′ monophosphate (cGMP) pathway prevented lysozyme induced vasdodilation. These inhibitors included methylene blue (Panel A), ODQ (1H-[1,2,4]oxadiazolo[4,3-a]quinoxalin-1-one) (Panel B), the protein kinase G inhibitor guanosine 3′,5′-cyclic monophosphorothioate, β-phenyl-1,N²-etheno-8-bromo-Rp-isomer, sodium salt (Panel C), and LY 83583 (6-anilino-5,8-quinolinequinone) (Panel D).

FIG. 6. In the example shown in Panel A, the peroxidase metabolizing enzyme Aspergillus niger catalase inhibited the effect of lysozyme. The mean results are shown in Panel B.

FIG. 7. In the example shown in Panel A, ethanol inhibited the effect of lysozyme. The mean results found at various ethanol concentration are shown in Panel B.

FIG. 8. In Panel A, the effect of H₂O₂ on force in the carotid preparation is shown. For the higher concentrations of H₂O₂, there was an initial increase in force that was followed by vasorelaxation. For the lower concentrations, vasorelaxation was produced. The mean results are shown in Panel B.

FIG. 9. Both the hydroxyl radical scavengers (mannitol) (Panel A) and hydroquinone (hyd) (Panel B) inhibited the vasodilatory effect of lysozyme in the carotid artery preparation.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

In septic shock, systemic vasodilation and myocardial depression contribute to the systemic hypotension observed. Both components reflect the effects of mediators that are released as part of the inflammatory response. We previously found that lysozyme (Lzm-S), released from leukocytes, contributed to the myocardial depression that develops in a canine model of septic shock. Lzm-S binds to the endocardial endothelium releasing nitric oxide (NO) that in turn activates the myocardial guanylate cyclase (sGC) pathway. In the present study, we determined whether Lzm-S may also contribute to the systemic vasodilation that is found in septic shock. In a phenylephrine contracted canine carotid artery ring preparation, we found that both canine and human Lzm-S, at concentrations similar to those found in sepsis, produced vasorelaxation. This relaxation could not be prevented by inhibitors of nitric oxide synthase, prostaglandin synthesis, or potassium channel inhibitors and was not dependent on the presence of the vascular endothelium. However, inhibitors of guanylate cyclase (sGC) pathway that included methylene blue, ODQ, LY83583, and a protein kinase G inhibitor all prevented Lzm-S's vasodilatory effect. In addition, the peroxide metabolizing enzyme, Aspergillus niger catalase, that would breakdown H₂O₂ as well as the hydroxyl scavengers that included hydroquinone and mannitol prevented Lzm-S's effect. These results are consistent with the notion that when Lzm-S interacts with the smooth muscle membrane, this interaction results in the formation of H₂O₂ that in turn activates the sGC pathway to cause vasorelaxation. This pathway may contribute to the vasodilation that occurs in septic shock.

Lzm-S is a newly discovered inflammatory mediator that has been shown to cause myocardial depression in an E coli experimental model of sepsis (20). In the present study, we examined the extent to which Lzm-S may contribute to the systemic vasodilation that is also observed in this model. The concentrations of Lzm-S used in this study were comparable to those previously found to occur after 4 to 6 hours of E coli bacteremia in dogs (20). In the present study, the results showed that both canine and human Lzm-S produced significant vasorelaxation in a phenylephrine contracted carotid artery ring preparation.

Based on our previous cardiac experiments (13, 19-21), we initially considered that the release of mediators from the endothelium, particularly NO, was the mechanism by which Lzm-S caused this vasodilatory effect. Nevertheless, when we tested for the role of NO by pre-treatment of the carotid ring with L-NMMA, we did not find that Lzm-S-induced vasorelaxation was inhibited. We further tested for commonly known pathways of vasodilation that included blockade of the prostaglandin pathway with indomethacin, blockade of the bradykinin β₁ and β₂ receptors by B9340, inhibition of adenyl cyclase production by SQ 22, 536, inhibition of potassium channels, as well inhibition by the β-adrenergic receptor antagonist propranolol and the muscarinic receptor antagonist atropine (6, 11,17, 30). In addition, when we removed the carotid artery endothelium, we found that the vasodilatory effect of Lzm-S was still present. Thus, the release of mediators from the endothelium or the aforementioned pathways did not contribute to the vasorelaxation caused by Lzm-S.

We also determined whether inhibitors of Lzm-S's catalytic site, namely chitobiose and chitotriose, would prevent Lzm-S-induced vasorelaxation (21,27). In the heart, the binding of Lzm-S to a N-glycoprotein on the endocardial endothelium mediates the release of NO (19). Lzm-S specifically binds to the Man β(1-4) GlcNAc β(1-4)GlcNAc moiety in the tri-mannosyl core structure of high mannose/hybrid and tri-antennary carbohydrate classes where GlcNAc is N-acetylglucosamine and Man is mannose (13). The Man β(1-4) GlcNAc β(1-4)GlcNAc moiety is structurally similar to chitobiose and chitotriose compounds, and these two agents have been shown to competitively inhibit Lzm-S's myocardial depressant effect in in-vivo and in-vitro preparations (13, 19-21), although the receptor to which Lzm-S binds is not clear. Thus, the signaling pathway and the critical binding site of Lzm-S with respect to vasorelaxation are distinctly different from the mechanisms that contribute to myocardial depression caused by this mediator.

Since removal of the endothelium did not inhibit Lzm-S's vasodilatory effect, the binding of Lzm-S to the smooth muscle cell rather than the endothelium resulted in the vasorelaxation observed. Moreover, this binding of Lzm-S to the smooth muscle membrane resulted in activation of the cGMP pathway, as evidenced by the fact that inhibitors of this pathway prevented Lzm-S's effect. Although activation of the cGMP pathway represented the apparent 2^{nd} messenger system for vasorelaxation, the mechanism by which this pathway was activated was not initially clear, since none of the commonly noted pathways, usually associated with cGMP activation, were able to block Lzm-S's effect. We therefore looked for other possibilities.

After many screening experiments were performed, we found that ethanol was able to inhibit Lzm-S's vasodilatory effect. We therefore focused on the experiments by the laboratory of Wolin and colleagues (2,3). In a precontracted bovine pulmonary artery preparation, these investigators showed that H₂O₂ mediates vasorelation and that both methylene blue and ethanol inhibited this effect. Ethanol is an agent that is known to modulate peroxide metabolism through endogenous catalase. Ethanol is associated with a decrease in the Compound I species that would in turn cause a reduction in cGMP thereby inhibiting vasodilation. In the present study, we hypothesized that the binding of Lzm-S to the smooth muscle cell might activate H₂O₂ and furthermore that this effect may be inhibited by both a peroxidase and ethanol. Since the results showed that both these agents blocked Lzm-S's vasodilatory effect, these findings would support the hypotheses of this study.

We also showed that exogenous H₂O₂ produced vasodilation in the carotid artery preparation. At the higher H₂O₂ concentrations that ranged between 10⁻³ to 10⁻¹ mol/L, there was a biphasic time response to H₂O₂ in which the initial response was a contraction at 5 minutes-post instillation that was followed by a vasodilator response at 30 minutes-post instillation. As concentrations of H₂O₂ were increased, we found that the relative magnitudes of both vasodilation and vasoconstriction increased, while at a concentration of 10⁻⁴ mol/L, the response was one of vasodilation alone. Burke and Wolin (2) also found a biphasic dose-response effect of exogenous H₂O₂ administration in a precontracted bovine pulmonary artery preparation, in which relative vasodilation increased as concentrations were raised from 10⁻⁶ to 10⁻⁴ mol/L after which vasorelaxation started to decrease at 10⁻³ mol/L. We think that both studies support the notion that exogenous H₂O₂ may activate multiple compartmental cellular pathways, since there were biphasic responses in both studies. Although the net effect on vasculature tone would depend on the extent to which each of these pathways was activated, the present study shows that Lzm-S produced a vasodilatory response in this carotid preparation.

We conclude that the present results are consistent with the notion that the interaction of Lzm-S with the vascular smooth muscle membrane results in the generation of H₂O₂ that in turn activates the cGMP pathway to cause vasorelaxation. Although generated extracellularly, H₂O₂ may initiate intracellular signaling pathways resulting in the activation of cGMP (8, 22). cGMP would then cause vasodilation by altering myofilament responsiveness to calcium or by inhibiting calcium entry into the cell (17).

The pathways by which H₂O₂ may be generated under different conditions are complex and not completely understood (5), and it is not yet clear the mechanism by which Lzm-S would generate H₂O₂. The potential enzymatic sources of reactive oxygen species, such as H₂O₂ include mitochondrial electron transport chain, the arachidonic acid metabolizing enzymes lipoxygenase and cycloxygenase, NAD(P)H oxidases, and other hemoproteins, among others. A membrane location for Lzm-S's site of H₂O₂ generation appears likely, since its 17,000 molecular weight would make transport to an intracellularly location less feasible. The majority of the bioactive H₂O₂ is derived from spontaneous or SOD catalyzed dismutation of O₂*⁻ (5). One electron reduction of molecular oxygen forms superoxide anion (O₂*⁻). Among the possible mechanisms to consider, we initially thought that H₂O₂ would be derived from superoxide with SOD, since this mechanism has been shown to activate guanylate cyclase in other experiments (7). However, we could not prevent Lzm-S's vasodilator effect with the SOD inhibitor DECTA, so that we do not think that this enzyme system was involved in the vasodilation observed.

Another mechanism to consider is that some proteins and antibodies may be capable of producing H₂O₂ under certain conditions. DeYulia et al (8) found that H₂O₂ could be generated by specific receptor-ligand interaction in cells and in cell-free systems. They showed that the extracellular domain of hematopoietin receptors interacting with cognate ligand is sufficient to generate H₂O₂, although the mechanism of this generation was not well defined. In other studies, Wentworth and colleagues (32-34) reported that proteins, such as antibodies may generate H₂O₂ from singlet molecular oxygen (¹O₂*) and water; moreover, that this generation may occur regardless of the antigen specificity of the antibody. They suggest that some proteins, particularly antibodies have the intrinsic ability to intercept ¹O₂* and efficiently reduce it to O₂*⁻ which then dismutates spontaneously into H₂O₂. Although the pathways have not been completely elucidated, we propose that a protein such as Lzm-S also processes the property to generate H₂O₂ in a manner described by Wentworth and colleagues (32-34). We further suggest that this is the initiating pathway by which vasodilation occurred in our preparation.

In the present study, we additionally showed that the hydroxyl radical scavengers mannitol and hydroquinone prevented Lzm-S's vasodilatory effect. Thus, in addition in H₂O₂, the formation of the hydroxyl radical appears to be an important component of the pathway that contributes to Lzm-S's effect. The blockade of Lzm-S-induced vasodilation by mannitol and hydroquinone was less effective (60% vs 90%) as compared to what was observed with the peroxidase from Aspergillus niger, so that the generation of the hydroxyl radical may play a lesser role than that of the H₂O₂ molecule in producing the vasodilation caused by Lzm-S. We also administered the iron chelator deferoxamine methanesulfonate salt that would prevent the conversion of H₂O₂ reaction to OH* and OH⁻ by the Fenton reaction. If the latter reaction were important to Lzm-S's vasodilatory effect, then we would have expected to find that the vasodilation by Lzm-S would be relatively inhibited. Wentworth et al also proposed that proteins could also generate OH* through a series of reactions that involved generation of trihydroxide radical resulting in the formation of ³O₂ and OH* and that this reaction may not require iron (35). Such a generation of OH* would be consistent with the pathway leading to the vasodilation caused by Lzm-S in the present study.

Although future studies are required to further elucidate the mechanism by which Lzm-S generates reactive oxygen species, this does not negate the significant findings of the present study showing that Lzm-S causes vasodilation by activation of the cGMP pathway, and that the initiating event appears to be the generation of H₂O₂ and OH*. Thus, in addition to its myocardial depressant activity, these results suggest that Lzm-S may contribute to the low systemic vascular resistance found in septic shock by a novel mechanism that involves the generation of reactive oxygen species. Burgess et al (4) showed that blood Lzm-S concentrations increase in patients with abdominal sepsis, so that we think that the findings in our canine models are relevant to what occurs in the human condition. Furthermore, in our previous studies, we showed that competitive inhibitors of Lzm-S's catalytic site, particularly chitotriose and chitobiose, were useful in the treatment of myocardial depression in canine models of septic shock (21), but in the present study, these agents could not prevent Lzm-S-induced vasorelaxation. A therapy that also targets against Lzm-S's vasodilator effect may therefore be of further benefit in the treatment of cardiovascular collapse in septic shock.

Thus, the inhibitors capable of reducing vasodilation caused by lysozyme may be administered to an individual in need of such treatment, for example, an individual undergoing vasodilation during sepsis or experiencing vasodilatory response due to other conditions or circumstances promoting production of H₂O₂ by lysozyme.

As will be appreciated by one of skill in the art, ‘reducing’ refers to a reduction in vasodilation compared to an untreated or mock treated control of similar condition. It is important to note that the control does not necessarily need to be repeated each time.

In one aspect of the invention, there is provided a method of reducing a vasodilatory response comprising administering to an individual in need of such treatment an effective amount of a guanylate cyclase pathway inhibitor or an H₂O₂ reducing agent.

As will be appreciated by one of skill in the art, ‘an effective amount’ refers to an amount that is sufficient to reduce vasodilation as discussed herein and will of course depend on the age, weight and condition of the individual in need of such treatment.

As discussed below, the guanylate cyclase pathway inhibitor may be for example but by no means limited to methylene blue, ODQ, LY83583, and a protein kinase G inhibitor.

The H₂O₂ reducing agent refers to an agent capable of reducing the levels of H₂O₂ and may be for example although by no means limited to Aspergillus niger catalase and hydroxyl scavengers.

The hydroxyl scavengers may be for example but are by no means limited to hydroquinone or mannitol.

Results

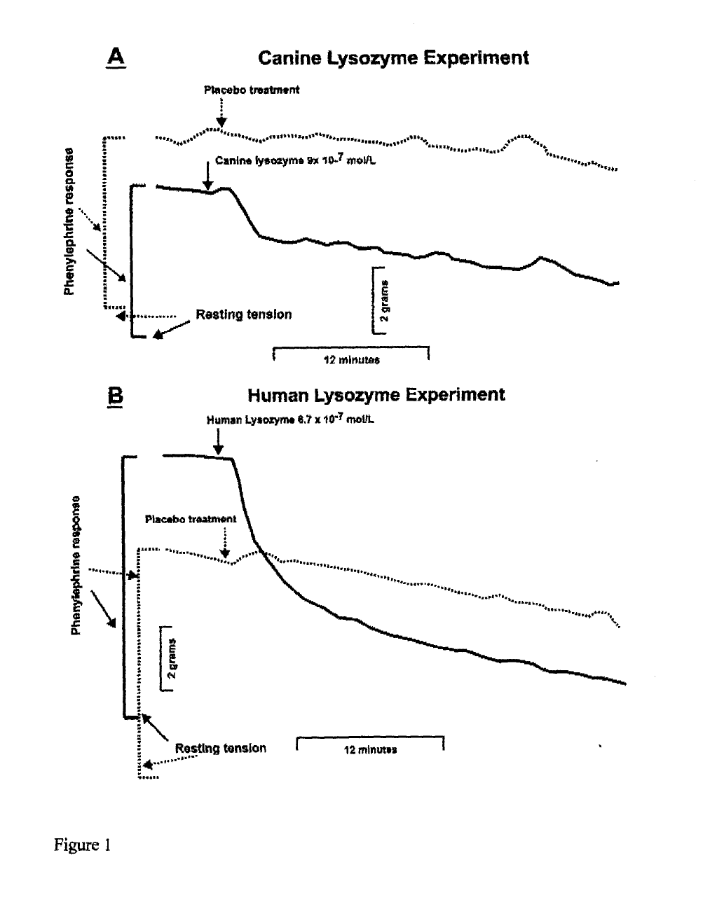

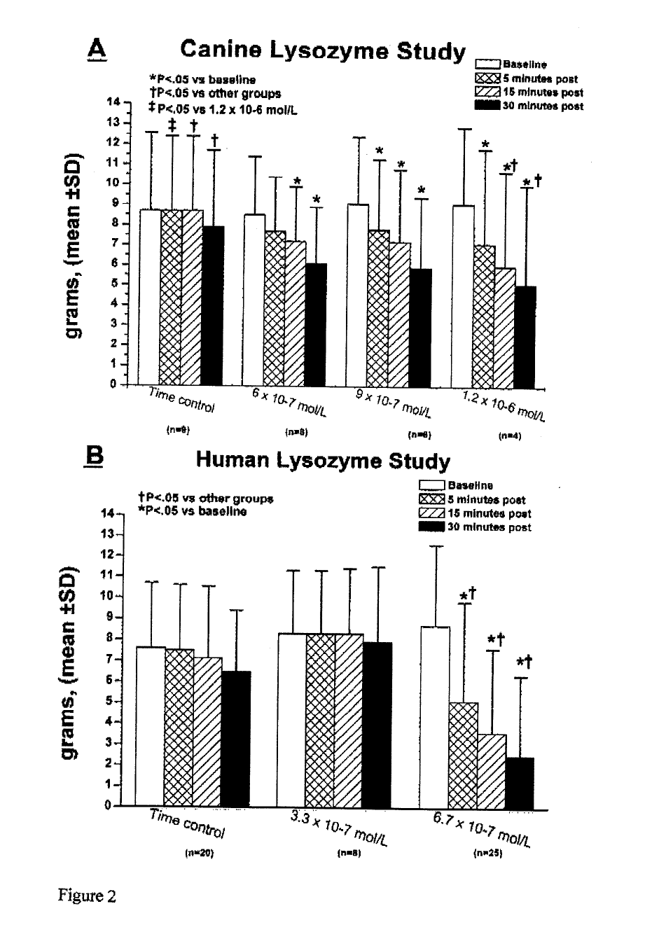

An example of the vasodilatory effect of canine Lzm-S is shown in Panel A in FIG. 1, while that of human Lzm-S is depicted in Panel B. In FIG. 2 (Panel A), the mean changes in contraction over the course of the study are shown. At a concentration of 1.2×10⁻⁶ mol/L, canine Lzm-S caused a decrease in contraction of approximately 50% at 30 minutes as compared with at pre-Lzm-S value, while there were smaller effects at the lesser concentrations. For the human Lzm-S experiment, phenylephrine also caused a similar increase in contraction among the three groups. As shown in FIG. 2 (Panel B), at a concentration of 3.3×10⁻⁷ mol/L, Lzm-S resulted in a minimal vasodilatory effect. For the higher concentration, however, there was a marked reduction in contraction in which Lzm-S nearly eliminated the increase in force induced by phenylephrine. Since human Lzm-S appeared to produce a large degree of vasorelaxation at 6.7×10⁻⁷ mol/L, this concentration of Lzm-S was used in the experiments described below.

Pretreatment with L-NMMA, Mechanical Removal of the Endothelium, Indomethacin, and Inhibitors of Lzm-S's Catalytic Site did not Prevent Vasodilation.

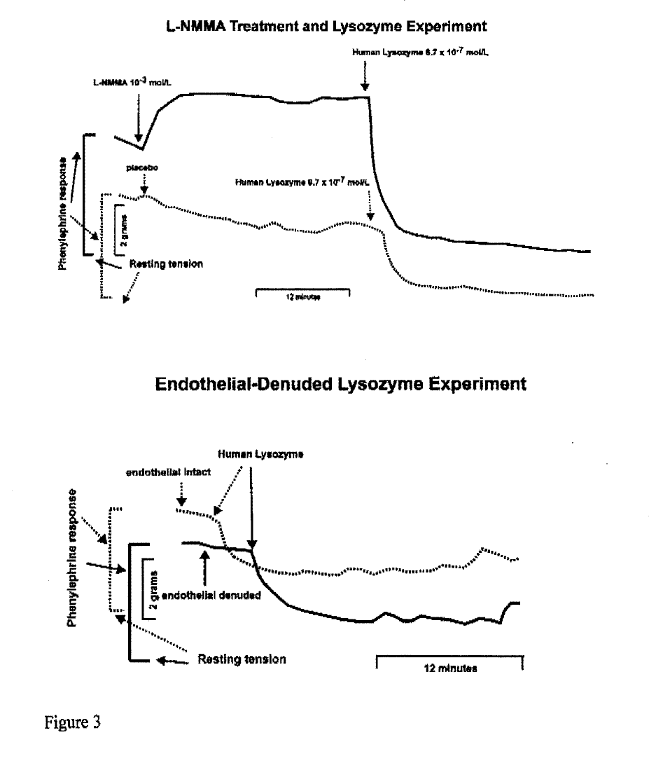

It was initially determined whether pretreatment with L-NMMA would prevent the vasodilatory effect of human Lzm-S. An example from these experiments is shown in FIG. 3, Panel A. In the L-NMMA treated group, phenylephrine caused a mean increase in resting force that averaged 6.2±2.4 grams, and contraction increased further to 8.1±3.5 grams with L-NMMA treatment. In the non-L-NMMA treated group, phenylephrine caused a mean increase in contraction from resting force of 7.1±2.1 grams, and contraction remained stable at 6.7±2.1 over the 30 minutes prior to Lzm-S administration. Despite the additional constriction observed with L-NMMA, Lzm-S produced a marked degree of relaxation that occurred to a similar extent in both the L-NMMA treated and non-treated groups. In the L-NMMA treated group (n=8) [relative to the value obtained post-L-NMMA treatment], Lzm-S caused a decrease in contraction to 37±22% * at 5 minutes, 13±16% * at 15 minutes, and −0.3±1% * at 30 minutes (the minus value indicates that contraction was below resting force) (*P<0.01 vs pre-Lzm-S plateau). In the non-L-NMMA treated group (n=12), relative to the value found pre-Lzm-S treatment, Lzm-S caused a decrease in contraction to 23±24% * at 5 minutes, 8±27% at 15* minutes, and 2±34% * at 30 minutes (*P<0.01 vs pre-Lzm-S plateau) [note, also, that in the statistical analysis, the results were compared in which force (in grams) rather than percentage declines were used in the analysis].

In the experiments performed in which the endothelium was mechanically removed, there was no inhibitory effect on Lzm-S's vasodilatory response. An example is shown in FIG. 3, Panel B. There was no difference in the phenylephrine response in the endothelial denuded and endothelial intact preparations (7.1±0.5 vs 6.7±0.7 grams). In the denuded preparation (n=4), the mean results showed that Lzm-S caused a decrease in contraction to 79±18% of the phenylephrine plateau at 5 minutes, 58±21% * at 15 minutes, and 54±20% * at 30 minutes (*P<0.05 vs phenylephrine plateau). In the intact preparation (n=4), Lzm-S caused a decrease in contraction to 64±25% of the phenylephrine plateau at 5 minutes, 53±33% * at 15 minutes, and 76±21% at 30 minutes (*P<0.05 vs phenylephrine plateau).

A similar lack of an effect was observed with indomethacin 10⁻⁶ mol/L treatment. In the indomethacin treated group, the mean increase in contraction from resting force with phenylephrine averaged 7.5±1.4 grams and force changed little over 30 minutes to 5.6±0.5 grams when indomethacin was added. In the non-indomethacin time control group, the mean increase in force from resting tension with phenylephrine averaged 6.9±2.7 grams and remained stable over 30 minutes at 6.1±1.8 grams. Despite indomethacin treatment, Lzm-S still produced marked vasodilation that occurred to a similar extent in both the indomethacin treated and non-treated groups. In the indomethacin treated group (n=5), relative to the post-indomethacin plateau, Lzm-S caused a decrease in contraction to 79±11% * at 5 minutes, 57±24% * at 15 minutes, and 39±25% * at 30 minutes (*P<0.01 vs pre-Lzm-S plateau). In the non-indomethacin treated group (n=4), relative to the pre-Lzm-S contraction, contraction decreased to 71±16% at 5 minutes, 45±19% * at 15 minutes, and 19±15% * at 30 minutes (*P<0.01 vs pre-Lzm-S plateau).

In addition, it was found that the competitive inhibitors of Lzm-S's catalytic site, namely chitobiose and chitotriose, did not block Lzm-S's vasodilatory response. The mean results are shown in Table 1.

Inhibitors of the cGMP Pathway Prevented Lzm-S-Induced Vasorelaxtion.

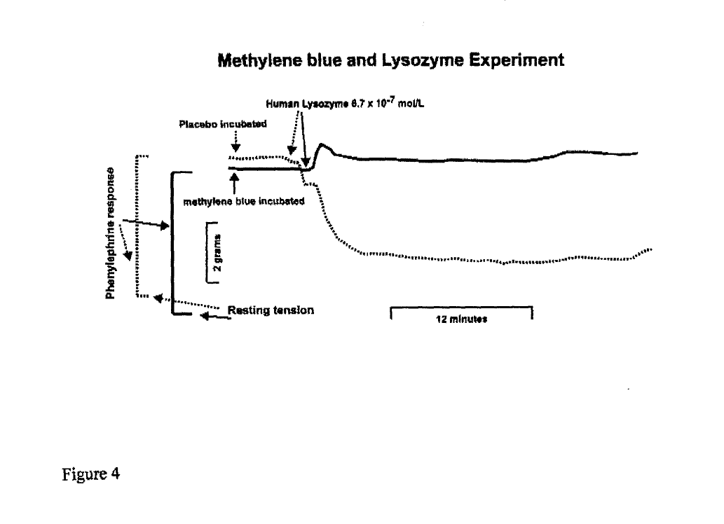

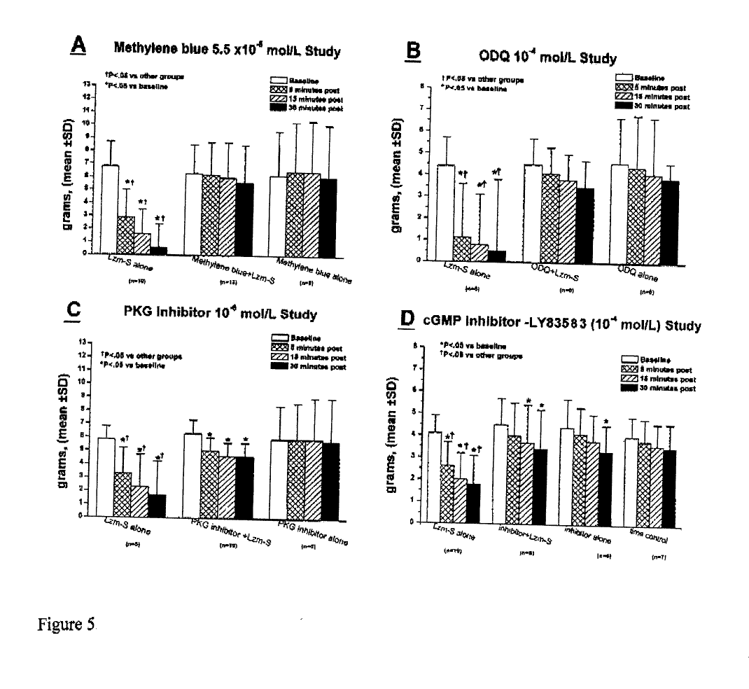

Since generation of cGMP, a known second messenger of vasodilation could be involved in Lzm-S's response, inhibitory compounds of this pathway were tested. These compounds included methylene blue (5.5×10⁻⁵ mol/L), LY 83583 (10⁻⁴ mol/L), ODQ (10⁻⁴ mol/L), and a PKG inhibitor (10⁻⁴ mol/L). All of these agents were found to inhibit Lzm-S's effect. An example for methylene blue is shown in FIG. 4. The mean results of the four inhibitors are shown in FIG. 5.

Agents that Modify H₂O₂ Metabolism also Inhibited the Vasodilation caused by Lzm-S.

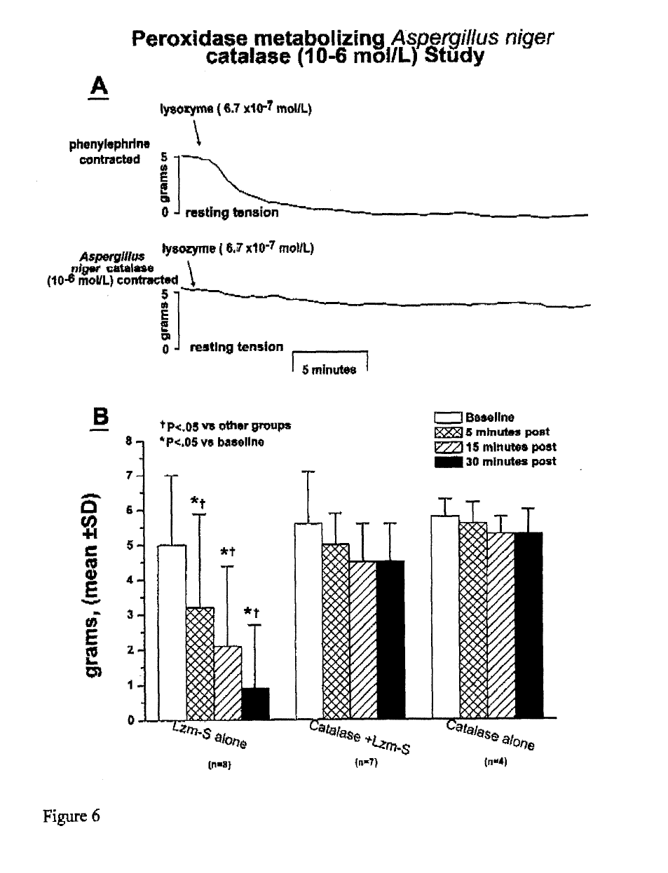

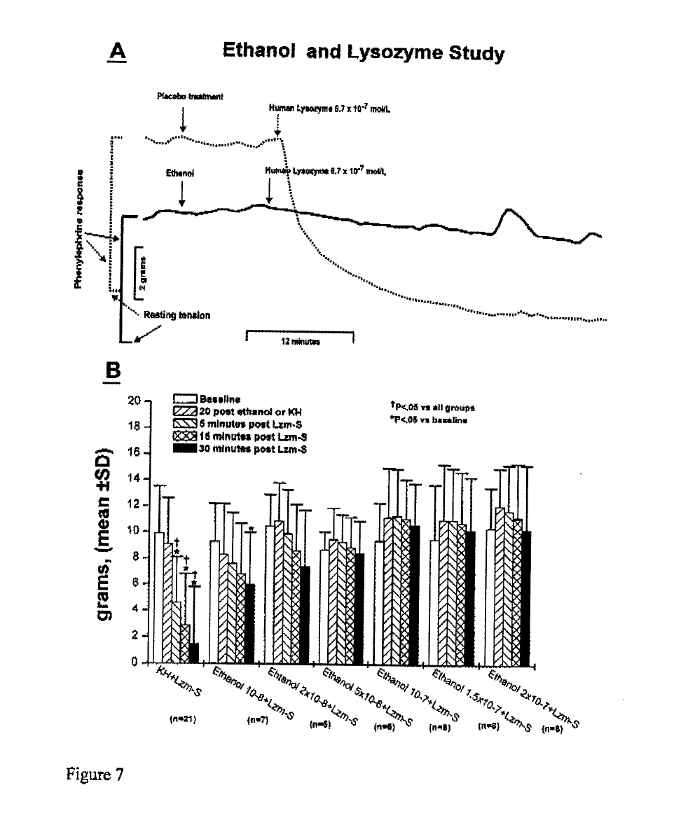

Based on the work of Wolin and colleagues (2, 3, 7), it was determined whether agents known to alter H₂O₂ metabolism might inhibit Lzm-S induced vasorelaxation. The peroxidase metabolizing enzyme, catalase from Aspergillus niger nearly completely inhibited the vasodilation due to Lzm-S (see Panels A and B in FIG. 6). Ethanol, which inhibits Compound I, was also markedly effective at the various concentrations that were used in prevention of Lzm-S-induced vasodilation (see FIG. 7). As shown in this figure, it was found that the higher concentrations of ethanol caused an initial vasoconstriction, but there was little change in force when Lzm-S was added to the preparation. In addition, in ethanol treated control groups, in which HEPES buffer rather than Lzm-S was added to the preparation, there was also no change in force with the buffer solution was added. In these control experiments, ethanol at the highest concentration (2×10⁻⁷ mol/L) caused a slight increase in contraction as compared with the phenylephrine contraction alone (8.2±2.6 grams to 10±2.6 grams; P<0.05 vs baseline), but force did not change further when HEPES buffer was added (10.7±2.8 grams at 5 minutes, 11±2.8 grams at 15 minutes, and 11±2.6 grams at 30 minutes).

Moreover, an unrelated alcohol, n-butanol, had no inhibitory effect on Lzm-S induced vasorelaxation. In contrast to ethanol, which caused an initial vasoconstriction, n-butanol caused an initial vasorelaxtion in which force decrease to 61±17% of the phenylephrine-plateau response after this alcohol was added (P<0.001 vs plateau response). The addition of Lzm-S caused further vasodilation to 84±9% of the butanol-plateau at 5 minutes, to 62±21% * at 15 minutes, and to 23±27% * at 30 minutes (*P<0.05 vs plateau response). These findings were not different from those observed in a Lzm-S treated group in which n-butanol treatment was not administered.

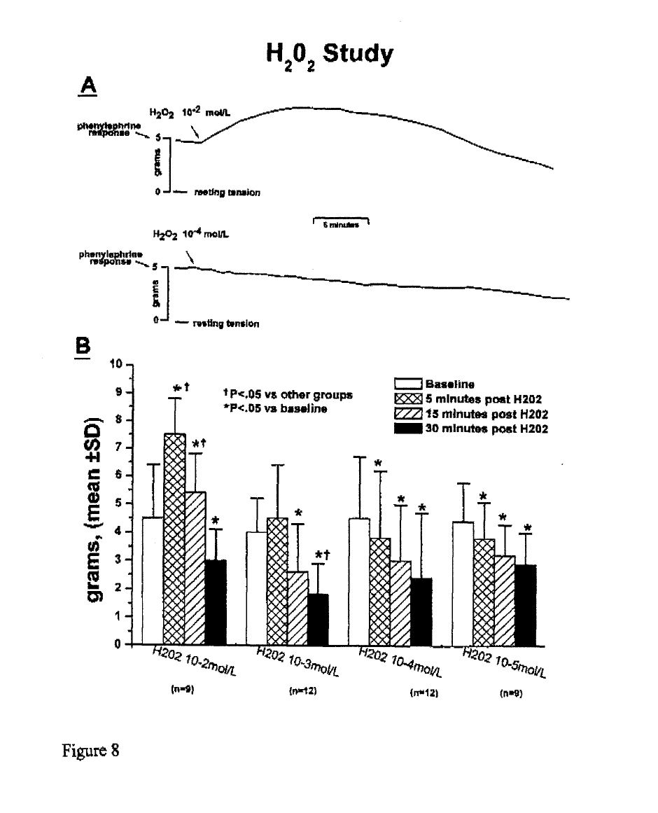

The effect of H₂O₂ on producing vasodilation is shown in FIG. 8. There was a biphasic time-effect on force at the different doses used. At concentrations between 10⁻³ to 10⁻¹ mol/L, the initial response was one of vasoconstriction that was rapidly followed by vasodilation. At a concentration of 10⁻⁴ mol/L, there was progressive vasorelaxation over time. At a concentration of 10⁻⁵ mol/L, there was a minimal effect of H₂O₂ on precontracted force.

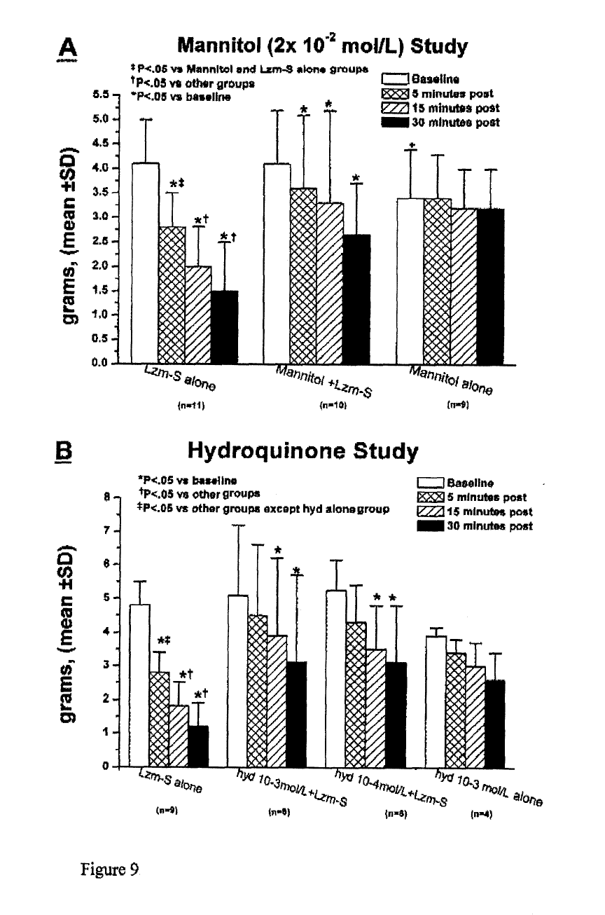

To determine whether the hydroxyl radical was involved in Lzm-S's effect, it was assessed whether the hydroxyl radical (OH*) scavengers, mannitol and hydroxyquinone (22), prevented Lzm-S induced vasorelaxation (see FIG. 9). Both treatments had an inhibitory effect.

In screening experiments (data not shown), neither DECTA (10⁻² mol/L), deferoxamine methanesulfonate salt (10⁻² mol/L), nor the reducing agents N-acetylcysteine (10⁻³ mol/L), ascorbic acid (10⁻⁴ mol/L), and reduced-glutathione (10⁻³ mol/L) were found to inhibit Lzm-S-induced vasodilation, so that additional experiments were not performed.

Methods

Carotid Artery Ring Preparation

Internal carotid arteries (4 cm length) were removed from mongrel dogs (15 to 25 kg) previously anesthetized with pentobarbital (45 mg/kg) (23,36). The arteries were placed in cold HEPES-buffered physiological solution (in mmol/L: 118 NaCl, 2.5 CaCl₂, 1.2 MgCl₂, 1.4 KH₂PO₄, 4.9 KCl, 25 HEPES and 11 glucose) bubbled with a 100% O₂. The arteries were dissected free from the surrounding tissues to obtain ≈4 mm diam rings. The rings were suspended in a 10 ml organ bath set at 37° with a pH of 7.35 by means of 2 stain-less steel triangles in which the ring is stretched at optimal length (to ≈4 g). The rationale for using the carotid vasculature rather than other systemic vessels was that more vasculature rings could be obtained from the carotid artery.

In all of the protocols described below, the carotid rings were preconstricted with phenylephrine (10⁻⁵ mol/L) (23). Measurements were determined at approximately 20 minutes post phenylephrine instillation when a stable plateau had been reached. After Lzm-S or placebo treatment was added to the preparation, measurements were obtained at 5 minutes, 15 minutes, and 30 minutes post instillation.

In initial experiments, it was determined the degree to which canine Lzm-S produced vasodilation in the precontracted carotid artery preparation. The plasma concentration of Lzm-S previously determined in our sepsis model is approximately 10⁻⁹ to 10⁻⁶ mol/L (20). The three concentrations of canine Lzm-S used in individual experiments were 6×10⁻⁷ mol/L, 9×10⁻⁷ mol/L, and 1.2×10⁻⁶ mol/L, respectively. The results obtained with Lzm-S were compared with those determined over an identical interval in which an equivalent amount of buffer solution was placed into the tissue bath. Canine Lzm-S was purified as previously described from the spleens of non-septic dogs by ARVYS Proteins, Inc (Stamford, Conn.) (20). The turbidimetric method of Shugar was used to confirm the presence of Lzm-S's enzymatic activity in the preparation (29). The purity of the preparation was determined by the finding of a single molecule species on MS/MS mass spectroscopy that was performed by WM Keck Foundation Biotechnology Resource Laboratory, Yale University (New Haven, Conn.).

In addition, the effect of human Lzm-S on producing vasodilation in the precontracted carotid artery ring preparation was examined. The objective was to determine whether human Lzm-S produced changes in vasomotor tone comparable to that found with canine Lzm-S. Human Lzm-S was purchased from EMD Biosciences (San Diego, Calif.). The two concentrations of human Lzm-S used in individual experiments were 3.3×10⁻⁷ mol/L and 6.7×10⁻⁷ mol/L, respectively. Although the degree to which vasodilation might occur was variable among the different batches of lysozyme bought from the same company, it should be noted that same batch was always used in all of the Lzm-S treated muscles in a given study.

To Determine whether NO Release, the Endothelium, or Prostanoids Contribute to Lzm-S-Induced Carotid Artery Vasodilation.

In one set of experiments, it was determined whether the release of NO by the carotid artery endothelium was responsible for the vasodilation caused by Lzm-S. Human Lzm-S was used in all of the experiments that follow, since the human preparation appeared to produce a more consistently potent vasodilatory response as compared with the canine preparation (see Results). In these NO experiments, after contraction was produced by phenylephrine, L-NMMA was added to the tissue bath at 10⁻³ mol/L (AG Scientific, Inc, San Diego, Calif.) for approximately 30 minutes to allow for maximal inhibitory effect (31). Immediately after instillation of L-NMMA, a vasoconstrictive response was noted that usually stabilized over the 30-minute interval. After stabilization was achieved, human Lzm-S at a concentration of 6.7×10⁻⁷ mol/L was added to the preparation. Measurements were expressed relative to the steady state contraction determined post-L-NMMA at 5 minutes, 15 minutes, and 30 minutes after Lzm-S instillation. The results were compared between L-NMMA treated and non-treated (HEPES buffer) groups over the aforementioned intervals.

In protocols analogous to those described for the L-NMMA experiments, it was also determined whether pretreatment with indomethacin (10⁻⁶ mol/L) to prevent prostaglandin release (14) and whether pretreatment with the traditional competitive inhibitors of Lzm-S [ie N,N′-diacetylchitobiose-chitobiose (10⁻³ mol/L) or N,N′,N″-triacetylchiotriose-chitotriose (10⁻³ mol/L)] (Sigma Corp, Oakville, Ont) (21,27) would prevent Lzm-S's vasodilatory effect.

After completion of the above studies, it was examined whether the carotid artery endothelium was necessary for Lzm-S-induced vasorelaxation. In these experiments, the endothelium was mechanically denuded from the carotid ring by means of a cotton-tipped applicator (2, 3, 7, 18). The vasomotor response to carbachol (10⁻³ mol/L) was used to determine whether the endothelium was successfully removed. Carbachol produces vasodilation in the intact preparation, while in the denuded preparation, vasoconstriction is predominantly observed. After denudation of endothelium was confirmed, the ring preparation was washed to remove the excess carbachol. After stabilization of the preparation, phenylephrine was then added to produce contraction. At the three predetermined intervals, the effect of Lzm-S was compared between the endothelial-denuded and intact preparations.

Since L-NMMA, indomethacin, the competitive inhibitors of Lzm-S (ie N,N′-diacetylchitobiose and N,N′,N″-triacetylchitotriose), as well as removal of the endothelium did not inhibit Lzm-S induced vasodilation (see Results), subsequent screening experiments were performed to examine whether other pathways may be involved. Among others, these studies included experiments that involved inhibition of the bradykinin β₁ and β₂ receptors by B9340 (11), inhibition of adenyl cyclase production by SQ 22, 536 (16), inhibition of potassium channels by a high KCl bath (30 mmol), 20-hydroxyeicosatetraenoic acid (30), tetraethylammonium, 4-aminopyridine, charybdotoxin and iberiotoxin (6), inhibition of β receptor activation by propranolol, inhibition of the muscarinic receptor by atropine, and inhibition of the histamine receptor by dipheniramine hydrochloride. Since none of these inhibitors prevented Lzm-S's vasodilatory response, other pathways were considered.

To Determine whether Activation of Soluble Form of Guanylate Cyclase (sGC) to Generate cGMP Contributes to Lzm-S-Induced Vasodilation

Since generation of cGMP, a known second messenger of vasodilation could be involved in Lzm-S's response (2,3), inhibitors of the cGMP pathway that included—LY 83583 (6-anilino-5,8-quinolinequinone, 10⁻⁴ mol/L, EMD, San Diego), ODQ (1H-[1,2,4]oxadiazolo[4,3-a]quinoxalin-1-one, 10⁻⁴ mol/L, EMD, San Diego Calif.), methylene blue (5.5×10⁻⁵ mol/L), and the protein kinase G inhibitor guanosine 3′,5′-cyclic monophosphorothioate, β-phenyl-1,N²-etheno-8-bromo-Rp-isomer, sodium salt (EMD, San Diego Calif.) (10⁻⁶ mol/L)—were examined (2, 3). In respective experiments, the carotid ring was incubated with one of these agents for approximately 30 minutes prior to phenylephrine induced contraction. After a stable phenylephrine response was achieved, the effect of human Lzm-S (6.7×10⁻⁷ mol/L) on inhibition of the vasodilatory response was determined at 5, 15, and 30 minutes post-instillation.

To Determine whether Lzm-S Induced Vasodilation may be Prevented by a Peroxidase Metabolizing Enzyme and Hydroxyl Radical Scavengers.

In precontracted isolated bovine intrapulmonary arterial rings, Burke and Wolin (2) showed that hydrogen peroxide (H₂O₂) produces a concentration dependent relaxation by a mechanism that is independent of the endothelium. They found that micromolar concentrations of H₂O₂ elicited increases in cGMP concentrations that were associated with decreases in force. This effect could be inhibited by prior treatment with the peroxidase metabolizing enzyme, catalase from Aspergillus niger. They further showed that H₂O₂ caused relaxation by interacting with endogenous catalase in which their results were most consistent with the involvement of Compound I, a species of catalase formed during the metabolism of endogenous peroxide, in the mechanism of cGC activation. This laboratory additionally showed that cGMP generation could be inhibited by ethanol that selectively interacts with Compound I (3) (see Discussion). In a rat liver preparation, Mittal and Murad (22) also considered that H₂O₂ could generate cGMP and proposed that hydroxyl radicals were also necessary for this generation to occur.

In our carotid artery preparation, the applicability of H₂O₂ production to Lzm-S-induced relaxation was determined within the framework of the experiments delineated by Wolin and colleagues (2,7). In one set of experiments, it was assessed whether ethanol (range: 10⁻⁸ mol/L to 2×10⁻⁷ mol/L) prevented Lzm-S's vasodilatory effect. The results obtained with ethanol were contrasted to those obtained when another alcohol-n-butanol (10⁻⁷ mol/L) was administered in order to assess whether this effect was specific for ethanol. It was also determined whether prior incubation of the carotid artery preparation with the peroxidase metabolizing enzyme, catalase from Aspergillus niger (10⁻⁶ mo/L, Sigma Corp, Oakville On) and whether the addition of the hydroxyl radical (OH*) scavengers (21) mannitol (10⁻³ mol to 10⁻¹ mol/L), and hydroxyquinone (10⁻³ mol/L to 10⁻⁴ mol/L) would inhibit Lzm-S-induced vasorelaxation.

In another experiment, it was examined whether H₂O₂ by itself (range: 10⁻⁵ mol/L to 10⁻¹ mol/L) would produce vasodilation in a manner similar to that found for Lzm-S. Finally, in respective experiments, it was assessed whether Lzm-S-induced vasodilation could be prevented by diethyldithiocarbamic acid (DECTA) which inhibits Zn⁺², Cu⁺² superoxide dismutase (SOD) (7), since superoxide generation could be important in Lzm-S's effect; by the iron chelator deferoxamine methanesulfonate salt (10⁻² mol/L) (5), which would prevent the conversion of H₂O₂ reaction to OH* and OH⁻ (by the Fenton reaction); and by reducing agents that included N-acetylcysteine (10⁻³ mol/L), ascorbic acid (10⁻⁴ mol/L), and reduced glutathione (10⁻³ mol/L).

Statistics. Differences in vasodilation among groups were determined by a two way (between—within ANOVA) analysis of variance. Student Newman Keuls' multiple comparison test was included to determine statistical differences between treatment groups when the ANOVA was used. In the statistical analysis, moreover, the results were compared in which force (in grams) rather than percentage declines were used in the analysis. In the design of the experiment, of the 8 to 10 rings obtained from a carotid artery of a given dog, each ring was used for a different subset of experiments in a specific study. Results are given as mean (±1 SD).

While the preferred embodiments of the invention have been described above, it will be recognized and understood that various modifications may be made therein, and the appended claims are intended to cover all such modifications which may fall within the spirit and scope of the invention.

### REFERENCES

1). Annane D, Bellissant E, Cavaillon J M. Septic shock. Lancet 365: 63-78, 2005.
2). Burke T M, Wolin M S. Hydrogen peroxide elicits pulmonary arterial relaxation and guanylate cyclase activation. Am J Physiol Heart Circ Physiol 252: H1267-H1273, 1987.
3). Burke-Wolin T M, Wolin M S. Inhibition of cGMP-associated pulmonary arterial relaxation to H₂O₂ and O₂ by ethanol. Am J Physiol Heart Circ Physiol 258: H1267-H1273, 1990.
4). Burgess P, Appel S H, Wilson C A, Polk H C. Jr. Detection of intraabdominal abscess by serum lysozyme estimation. Surgery 115: 16-21, 1994.
5). Cai H. Hydrogen peroxide regulation of endothelial function: origins, mechanisms, and consequences. Cardiovascular Research 68: 26-36, 2005.
6). Calderone V, Martelli A, Testai L, Martinotti E, Breshi M C. Functional contribution of the endothelial component to the vasorelaxing effect of resveratrol and NS 1619, activators of the large-conductance calcium-activated potassium channels. Naunyn Schmiedebergs Arch Pharmacol 375: 73-80, 2007.
7). Cherry P D, Omar H A, Farrell A, Stuart J S, Wolin M S. Superoxide anion inhibits cGMP-associated bovine pulmonary arterial relaxation. Am J Physiol Heart Circ Physiol 259: H1056-H1062, 1990.
8). DeYulia G J, Carcamo J M, Borquez-Ojeda O, Shelton C C, Golde D W. Hydrogen peroxide generated extracellularly by receptor-ligand interaction facilitates cell signaling. PNAS 102: 5044-5049, 2005.
9). Fleming A. On a remarkable bacteriolytic element found in tissue and secretions. Proc R Soc Lond (Biol) 93: 306-307, 1922.
10). Grover R, Zaccardelli D, Colice G, Guntupalli K, Watson D, Vincent J L. An open label dose escalation study of the nitric oxide synthase inhibitor, N(G) methyl-L-arginine hydrochloride (546C88) in patients with septic shock. Glaxo Wellcome International Septic Shock Study Group. Crit Care Med 27:913-922, 1999.
11). Helmy A, Ferguson J W, Hayes P C, Newby D E, Webb D J. Bradykinin does not contribute to peripheral vascular tone in patients with cirrhosis and ascites. J Cardiovasc Pharmacol 47:556-60, 2006.
12). Hotchkiss R S, Karl I. The pathophysiology and treatment of sepsis. N Eng J Med 348: 138-148, 2003.
13). Jacobs H, Mink S N, Duke K, Bose D, Cheng, Z Q, Howlett S, Ferrier G R, Light R B. Characterization of membrane N-glycan binding sites of lysozyme for cardiac depression in sepsis. Intensive Care Medicine. 31:129-137, 2005.
14). Kato M, Nishida S, Kitasato H, Sakata N, Kawal S. Cyclooxygenase-1 and cyclooxygenase-2 selectivity of non-steroidal anti-inflammatory drugs: investigation using human peripheral monocytes. J Pharm Pharmacol 53:1679-85, 2001.
15). Kwok E S H, Howes D. Use of methylene blue in sepsis: a systematic review. J Intensive Care Med 21: 359-363, 2006.
16). Lippe C, Ardizzone C. Actions of vasopressin and isoprenaline on the ionic transport across the isolated frog skin in the presence and the absence of adenyl cyclase inhibitors MDL 12330 and SQ22536. Comp Biochem Physiol 99: 209-11, 1991.
17). Lohmann S M, Vaandrager A B, Smolenski A, Walter U, De Jonge H R Distinct and specific functions of cGMP-dependent protein kinases. Trends Biochem. Sci 22: 307-312, 1997.
18). Mathew R, Omar H A, Cherry P D, Gewitz M H, Wolin M S. Role of cGMP mechanism in response of rat pulmonary arteries to hypoxia. Am J Physiol Heart Circ Physiol 263: H141-H146, 1992.
19). Mink S N, Bose R, Roberts D E, Jacobs H, Duke K, Bose D, Cheng Z Q, Light R B. Lysozyme binding to endocardial endothelium mediates myocardial depression by the nitric-oxide guanosine 3′,5′ monophosphate pathway in sepsis. J Mol Cellular Card 39: 615-625, 2005.
20). Mink S N, Jacobs H, Bose D, Duke K, Cheng Z Q, Liu G, Light R B. Lysozyme: a mediator of myocardial depression and adrenergic dysfunction in septic shock in dogs. J Mol Cellular Card 35: 265-275, 2003.
21). Mink S N, Jacobs H, Duke K, Bose D, Cheng Z Q, Light R B. N,N′,N″ triacetylglucosamine, an inhibitor of lysozyme, prevents myocardial depression in E coli sepsis in dogs. Critical Care Med 32: 184-193, 2004.
22). Mital C K, Murad F. Activation of guanylate cyclase by superoxide dismutase and hydroxyl radical: a physiological regulator of gunaosine 3′,5′ monophosphate formation. PNAS 74: 4360-4364, 1977.
23). Mitsumizo S, Nakashima M, Hamada T, Totoki T. NOS II inhibition restores attenuation of endothelium-dependent hyperpolarization in rat mesenteric artery exposed to lipopolysaccharide. J Cardiovasc Pharmacol 43:589-94, 2004.
24). National Institutes of Health. Guide for the Care and Use of Laboratory Animals (1996) Bethesda, Md., National Institutes of Health, (NIH Publication No 85-23).
25). Parker M M, Shelhamer J H, Bacharach S L, Green M V, Natanson C, Frederick T M, Damske B A, Parrillo J E. Profound but reversible myocardial depression in patients with septic shock. Ann Intern Med 100: 483-490, 1984.
26). Parrillo J E, Burch C, Shelhamer J H, Parker M M, Natanson C, Schuette W. A circulating myocardial depressant substance in humans with septic shock. J Clin Invest 76: 1539-1553, 1985.
27). Rand-Meir T, Dahlquist F W, Raftery M A. Use of synthetic substrates to study binding and catalysis by lysozyme. Biochemistry 8: 4206-4214, 1969.
28). Rice T W, Bernard G R. Therapeutic intervention and targets for sepsis. Annu Rev Med 56:225-248, 2005.
29). Shugar D. Measurement of lysozyme activity and the ultra violet inactivation of lysozyme. Biochim Biophys Acta 8:302-309, 1952.
30). Tanaka Y, Koike K, Toro L. MaxiK channel roles in blood vessel relaxations induced by endothelium-derived relaxing factors and their molecular mechanisms. J Smooth Muscle Res 40: 125-153, 2004.
31). Tatsumi T, Keira N, Akashi K, Kobara M, Matoba S, Shiraishi J, Yamanaka S, Mano A, Takeda M, Nishikawa S, Asayama J, Fliss H, Nakagawa M. Nitric oxide-cGMP pathway is involved in endotoxin-induced contractile dysfunction in rat hearts. J Appl Physiol 96:853-860, 2004.
32). Wentworth A D, Jones L H, Wentworth P, Jr, Janda K D, Lerner R A. Antibodies have the intrinsic capacity to destroy antigens. PNAS 97: 10930-10935, 2000.
33). Wentworth P, Jr, Hones L H, Wentworth A D, Zhu X, Larsen N A, Wilson I A, Xu X, Goddard W A, Janda K D, Eschenmoser A, Lerner R A. Antibody catalysis of the oxidation of water. Science 293: 1806-1811, 2001.
34). Wentworth P, Jones L H, Wentworth A D, Zhu X, Larsen N A, Wilson I A, Xu X, Goddard W A, Janda K, Eschenmoser A, Lerner R A. Antibody catalysis of the oxidation of water. Science 293: 1806-1811, 2001.
35). Wentworth P, Wentworth A D, Zhu X, Wilson I A, Janda K D, Eschenmoser A, Lerner R A. Evidence for the production of trioxygen species during antibody-catalyzed chemical modification of antigens. PNAS 100: 1490-1493, 2003.
36). Yu J, Guia A, Mink S, Kepron W, Cragoe E J, Sharma, Bose R. Role of sodium in antigen-induced contraction of tracheal smooth muscle in dogs. Respir Physiol 91:111-24, 1993.

1. A method of reducing a vasodilatory response comprising administering to an individual in need of such treatment an effective amount of a guanylate cyclase pathway inhibitor or an H₂O₂ reducing agent.

2. The method according to claim 1 wherein the guanylate cyclase pathway inhibitor is selected from the group consisting of methylene blue, ODQ, LY83583, and a protein kinase G inhibitor.

3. The method according to claim 1 wherein the H₂O₂ reducing agent is selected from the group consisting of: Aspergillus niger catalase and hydroxyl scavengers.

4. The method according to claim 3 wherein the hydroxyl scavengers are hydroquinone or mannitol.
